(19) United States
(12) Patent Application Publication
(10) Pub. No.: US 2019/0351278 A1
(43) Pub. Date: Nov. 21, 2019

(54) COMPRESSION LEGGINGS WITH ATTACHED RESISTANCE BANDS

(71) Applicant: Jacqueline Nicole Walker, Jacksonville, FL (US)

(72) Inventor: Jacqueline Nicole Walker, Jacksonville, FL (US)

(21) Appl. No.: 15/980,911

(22) Filed: May 16, 2018

(51) Int. Cl. A63B 21/00; A41D 1/08; A61F 13/08; A63B 21/04; A63B 21/055; A63B 23/035; A63B 23/04

(52) U.S. Cl. CPC A63B 21/4011; A41D 1/08; A61F 13/08; A63B 21/0428; A63B 2023/006; A63B 23/03516; A63B 23/0488; A41D 2300/322; A41D 2600/10; A63B 21/0557

(57) ABSTRACT

Compression and moisture wicking leggings include attached resistance bands on a single leg. The resistance band is stretched and wrapped around the second leg for a resistance-based workout. The leggings also include pockets for inserting weights for an increased intensity workout.

### BACKGROUND OF THE INVENTION

The current invention relates to compression devices for exercise, and more particularly, to compression and moisture wicking leggings with two attached resistance bands.

The lines that draw the intersection between fitness wear, sports apparel, and leisurewear are slowly disappearing and the difference between active wear, casual wear, fitness clothing, and sportswear are merging, thus changing the target audience as well. There is anticipated growth in the market due to the rise in adventure sports, growing geriatric population, and a rising obese population as people shift their focus towards healthy living.

People often use resistance bands as part of their exercise routine. Closed circuit resistance bands often roll up the wearer's leg. Further, people often forget their resistance bands at home. Closed circuit resistance bands offer a great amount of utilization but is limited due to proper attire and potential user error. For example, resistance bands tend to work better on skin rather than certain fabrics. When using resistance bands, often skin is in contact with the band causing stickiness and discomfort. Existing resistance bands are hard to put over larger thighs and often roll up or down.

There exists a need for apparel with built in resistance bands for ease of use and the ability for utilization at any time.

### BRIEF DESCRIPTION OF THE DRAWINGS

FIG. 1 is one embodiment of a front right perspective view of the invention wrapped around a single leg;

FIG. 2 is one embodiment of a rear left perspective view of the invention wrapped around a single leg;

FIG. 3 is a detail rear left perspective view of the invention;

FIG. 4 is one embodiment of a front right perspective view of the invention wrapped around both legs;

FIG. 5 is one embodiment of a rear left perspective view of the invention wrapped around both legs;

FIG. 6 is one embodiment of a front right exploded view of the invention demonstrating weight insertion regions;

FIG. 7 is one embodiment of a detail front right perspective view of the invention;

FIG. 8 is a section detail view of the invention taken along line 8-8 in FIG. 2;

FIG. 9 is a section detail view of the invention taken along line 9-9 in FIG. 7 and shown with weight insertion;

FIG. 10 is a detail front left perspective view of the first alternate embodiment of the invention;

FIG. 11 is a detail front left perspective view of the first alternate embodiment of the invention;

FIG. 12 is a detail front right perspective view of the first alternate embodiment of the invention;

FIG. 13 is a detail front right perspective view of the first alternate embodiment of the invention;

FIG. 14 is a section detail view of the first alternate embodiment of the invention taken along line 14-14 in FIG. 12;

FIG. 15 is a section detail view of the first alternate embodiment of the invention taken along line 15-15 in FIG. 13;

FIG. 16 is a section detail view of the invention taken along line 16-16 in FIG. 11;

FIG. 17 is a front right perspective view of the second alternate embodiment of the invention; and

FIG. 18 is a front right perspective view of the second alternate embodiment of the invention.

### SUMMARY

A wearable exercise system includes a pair of leggings configured to be worn by a user, the pair of leggings having a first leg and a second leg, and a stretchable resistance band attached around the first leg of the pair of leggings, the stretchable resistance band being attached on a thigh area of the first leg. The exercise system further includes a closure mechanism attached to the stretchable resistance band, wherein the closure mechanism is configured to open and close a loop of the stretchable resistance band, the stretchable resistance band is configured to be opened and stretched around the second leg of the pair of leggings, and the closure mechanism closes the loop of the stretchable resistance band around the second leg of the pair of leggings while worn by the user.

In one embodiment, the stretchable resistance band is attached about 1.5 inches above a knee area of the pair of leggings.

In one embodiment, a second stretchable resistance band is attached about 1.5 inches above an ankle area of the first leg of the pair of leggings. The second stretchable resistance band is configured to be opened, stretched around the second leg of the pair of leggings, and closed around the second leg while worn by the user.

In one embodiment, a layered stretchable resistance band is attached to the pair of leggings under the stretchable resistance band. The layered stretchable resistance band is configured to be opened, stretched around the second leg of the pair of leggings, and closed around the second leg while worn by the user.

In one embodiment, the exercise system includes a plurality of stretchable resistance bands attached around the first leg of the pair of leggings. In one embodiment, the first leg is a right-side leg and the second leg is a left side leg. In one embodiment, the first leg is a left side leg and the second leg is a right-side leg.

In one embodiment, a vertical section of the stretchable resistance band is attached with a seam to the pair of leggings.

In one embodiment, the closure mechanism is a zipper. In one embodiment, the pair of leggings further comprises at least one legging loop, wherein one band of the stretchable resistance band fits through the legging loop. In one embodiment, the exercise system comprises at least one pocket attached to the pair of leggings, the pocket configured to hold a weight.

These and other features, aspects and advantages of the present invention will become better understood with reference to the following drawings, description and claims.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

As stated above, closed circuit resistance bands have many limitations. The invention claimed here solves this problem.

Broadly, one embodiment of the present invention is a pair of leggings with attached resistance bands. The bands advantageously do not roll up the user's legs. The user will not forget their resistance bands, because they are wearing them. The leggings allow the user to have a quick workout anywhere. Additionally, closed circuit resistance bands utilization is increased due to their attachment to the leggings, thus being ideal for walking, running, biking, and other plyometric exercises. The resistance bands do not touch the skin, and thus, do not increase sweat and/or discomfort for the user. Further, there is a wider range of resistance with the bands attached.

The claimed invention differs from what currently exists. Current compression clothing does not allow the user to transform the clothing for when they do and do not want to work out. Advantageously, there is discretion in the described leggings. Beneficially, the user transforms everyday leggings into fitness gear at a moment's notice. The resistance bands are not difficult to “put on,” because they are already attached.

The described leggings are high quality fitness attire that have built in complexities that allow for an optimal exercise routine. Advantageously, they can be worn by anyone wishing to increase the quality of their fitness regimen. The leggings and accompanying features have been designed for both genders and may be worn by any age. The leggings are crafted to aide in increasing the ease of use for resistance bands while allowing for a larger variety of exercise options. They provide the ability to build muscle, provide body toning, and enhance weight loss.

As shown in the embodiment of FIG. 1, the invention includes a pair of moisture wicking leggings 12 with a plurality of legging loops 14. In one embodiment, the moisture wicking leggings 12 include eight legging loops 14; one on a front and back thigh on each leg and one on a back and front calf of each leg. At least one resistance band 22 is positioned through a legging loop 14. In one embodiment, the moisture wicking leggings are made from moisture wicking and compression fabrics such as polyester, spandex, nylon, or other suitable materials.

In one embodiment, one resistance band 22 is in the thigh area about 1.5 inches above the knee, and one resistance band 22 is about 1.5 inches above the ankle. The placement of the resistance bands 22 allows for increased muscle activation and provides ease of use. Advantageously, the location of the resistance bands 22 decrease the likelihood of injury and increase muscle activation.

Resistance bands 22 include band zippers 16 to zip and unzip the resistance bands 22. Leggings 12 further include a plurality of pockets 18. In one embodiment, the resistance bands 22 are made of fabrics such as elastodien, polyester, polyamide, or other suitable resistance type fabrics.

In an alternative embodiment, the resistance band 22 can include any suitable closure mechanism and is not limited to only zipper 16.

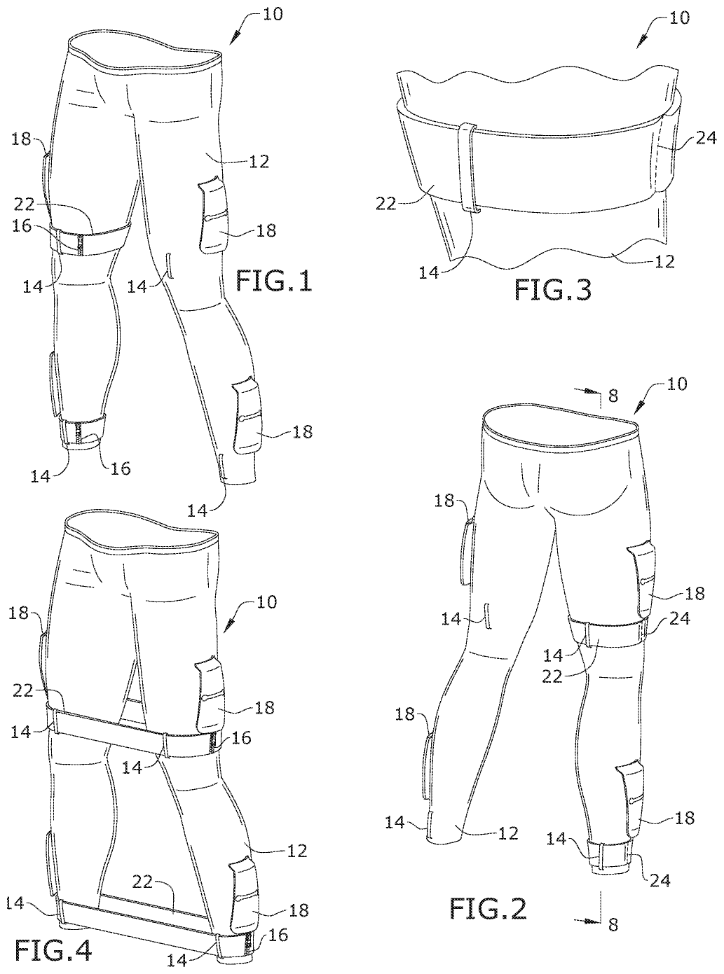

FIG. 2 shows a rear view of leggings 12. The resistance bands 22 are shown with seams 24, where the resistance bands 22 are attached to leggings 12 by sewing or other attachment means. FIG. 3 shows a detailed perspective view of the resistance band 22 with seam 24.

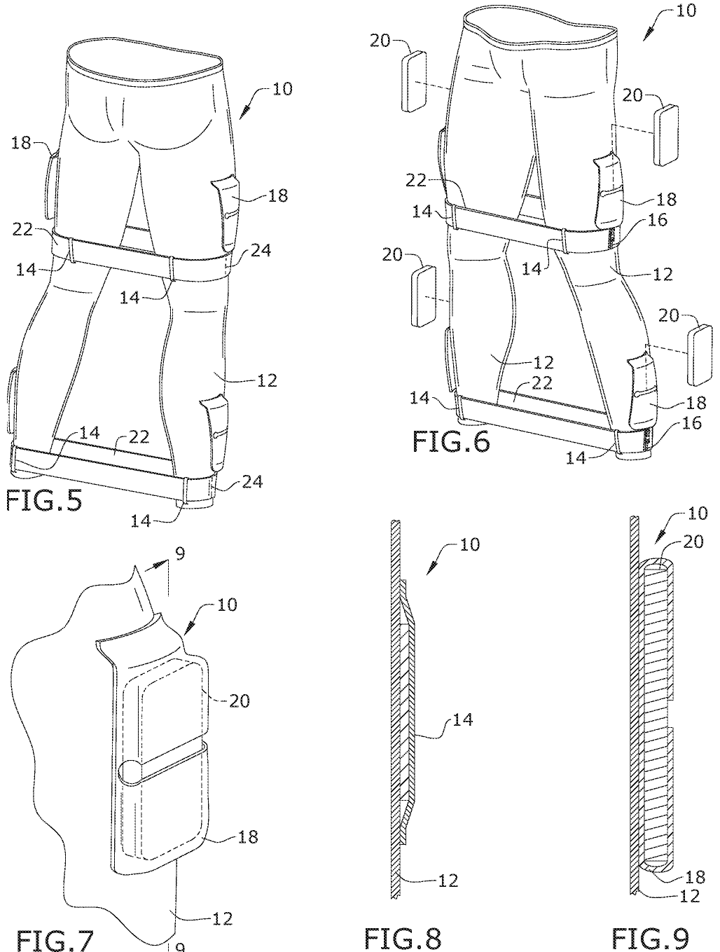

The resistance bands 22 on each leg wrap around the leg itself. As shown in the embodiment of FIG. 4, the resistance bands 22 are unwrapped by unzipping the zipper 16, stretching each end, wrapping the ends around the other thigh, and zipping the ends together with zipper 16. The user utilizes the resistance bands 22 how they see fit without worry of the resistance bands 22 rolling up or down. FIG. 5 shows a rear view of the leggings 12 as the bands 22 are wrapped around both legs.

In one embodiment as shown in FIG. 6, a weight 20 is optionally inserted into the pocket 18. The wearer can insert the weights 20 in one pocket 18, all the pockets 18, or in none of the pockets 18. The weights 18 can be of any desirable weight. FIG. 7 shows a detailed right perspective view the weight 20 inserted in pocket 18.

FIG. 8 shows a section view taken along line 8-8 of FIG. 2. The band 14 is shown on leggings 12. FIG. 9 shows a section detail view taken along line 9-9 of FIG. 7. It shows weight 20 inserted into pocket 18 on leggings 12.

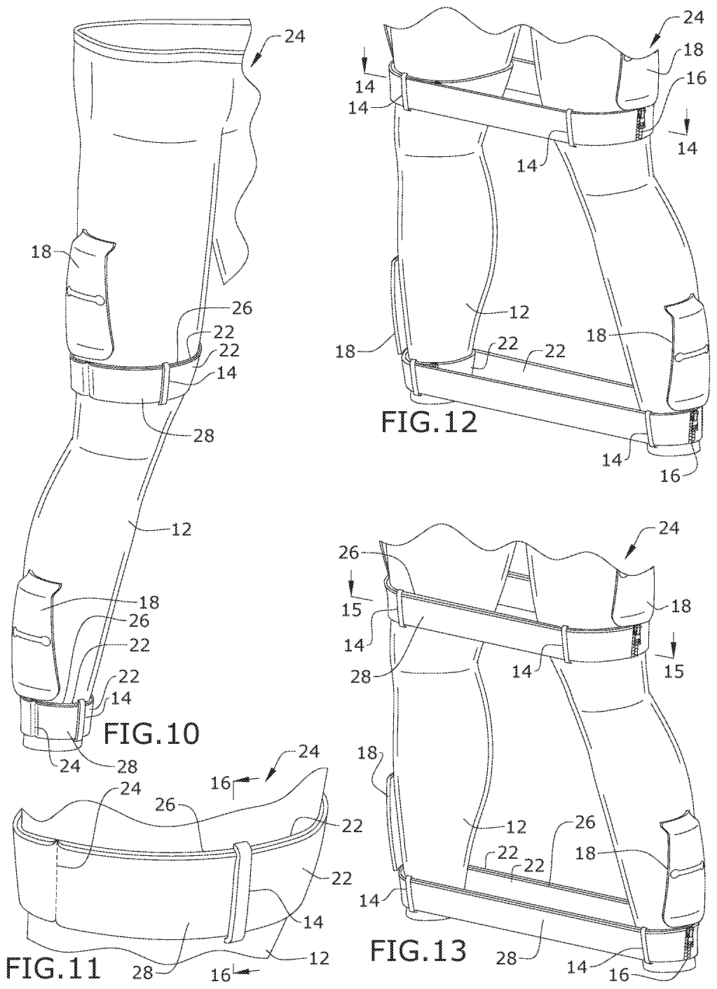

FIG. 10 shows an alternative embodiment of the invention with layered resistance bands. The first resistance band 22 is layered on top of a lower resistance band 26 on the upper thigh, and the ankle resistance band 22 is layered on the second lower resistance band 26. The two upper resistance bands 22 and the two lower resistance bands 26 are inserted through the legging loops 14. The lower resistance bands 26 also include zippers 16 to open and close the lower resistance bands 26, independent of resistance bands 22.

FIG. 11 shows a detailed view of the layered resistance bands 22 and 26 on a single leg. The bands 22 and 26 are sewn together at seam 24. FIG. 12 shows one embodiment where only the upper resistance bands 22 are stretched and wrapped around the other leg. FIG. 13 shows an embodiment wherein both upper resistance bands 22 and lower resistance bands 26 are unwrapped, stretched across both legs, and zipped to create an increased resistance.

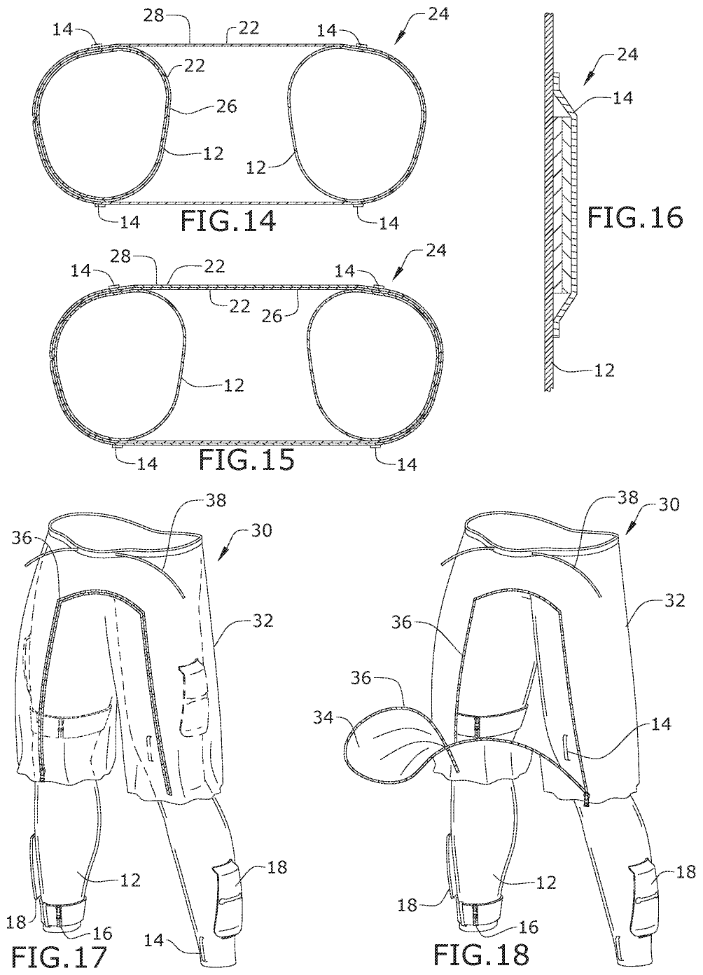

FIG. 14 shows a section view taken along line 14-14 of FIG. 12, wherein only the upper resistance bands 22 are used to create resistance. FIG. 15 shows a section view taken along line 15-15 of FIG. 13, wherein both upper resistance bands 22 and lower resistance bands 26 are used to create increased resistance.

FIG. 16 shows a section detail view of the invention taken along line 16-16 of FIG. 11, showing the upper resistance bands 22 and lower resistance bands 26, positioned through band loop 14.

FIG. 17 shows an alternative embodiment comprising a pair of shorts 32 attached to the leggings 12. The shorts 32 include a draw string 38 and a shorts flap 34 with a shorts flap zipper 26. The shorts flap zipper 26 is unzipped to access the thigh resistance bands 22. Weights 20 can be added to pockets 18 if the wearer desires, to increase the workout intensity. Advantageously, the shorts 32 are suitable for males, females, or anyone not wishing to draw attention to curvy body parts or being overweight.

Advantageously, the resistance bands 22 and 26 are easily washable, while allowing for increase ease of use and discretion in fitness gear. The leggings 12 aid in wicking away moisture from the user when working out. The compression of the leggings 12 also allows for support while the user is working out. The band loops 14 hold the resistance bands 22 and 26 in place, so that they will not roll down while being used. The resistance bands 22 and 26 are in place for use while sitting, standing, and performing various workouts.

In one embodiment, to make the leggings 12 with resistance bands 22, a compression fabric, moisture wicking fabric, and washable resistance bands are obtained. On a left and right outer seam of the leggings 12, the resistance bands 22 are attached to the legging material.

In one embodiment, one resistance band 22 is attached high on the thigh, and one is below mid-calf. The resistance band 22 is attached in a manner that allows the resistance band 22 to be wrapped around each perspective section of the leg. It is to be understood that in alternative, additional resistance bands 22 and lower resistance bands can be added along different areas of the leg for additional resistance. Further, any suitable number of legging loops 14 can be added or taken away on the leggings 12.

Working out can inadvertently cause a person to be more self-conscious. When using workout equipment that malfunctions, the user may be less inclined to use or may use improperly. The leggings 12 are advantageously user friendly and increase utilization. The leggings 12 may help in increasing muscle tone when doing squats. For example, the user can unwrap the thigh band 22, attach to the other thigh, and then do squats. To increase or change resistance, the user can use the bands 22 and 26 in any combination of ways they deem fit. In additional, the user can add one or more weights 20 into the pockets 18.

The leggings 12 are used by anyone with the goal for optimal workout experience, specifically working the lower body muscle groups. The leggings 12 allow for both male and female to benefit from using resistance bands without having to worry about placement of the bands, user error, or physical inexperience. These leggings are not only designed to incorporate a higher level of fitness, but also to deliver comfort when working out. The compression and moisture wicking fabrics prevent the feeling of sweat and will keep the body tight.

It should be understood, of course, that the foregoing relates to exemplary embodiments of the invention and that modifications may be made without departing from the spirit and scope of the invention as set forth in the following claims.

What is claimed is:

1. A wearable exercise system, the system comprising:
a pair of leggings configured to be worn by a user, the pair of leggings having a first leg and a second leg;
a stretchable resistance band attached around the first leg of the pair of leggings, the stretchable resistance band being attached on a thigh area of the first leg; and
a closure mechanism attached to the stretchable resistance band, the closure mechanism configured to open and close a loop of the stretchable resistance band, the stretchable resistance band configured to be opened and stretched around the second leg of the pair of leggings, the closure mechanism closing the loop of the stretchable resistance band around the second leg of the pair of leggings while worn by the user.

2. The wearable exercise system of claim 1, wherein the stretchable resistance band is attached about 1.5 inches above a knee area of the first leg.

3. The wearable exercise system of claim 1 further comprising a second stretchable resistance band being attached about 1.5 inches above an ankle area of the first leg of the pair of leggings, the second stretchable resistance band configured to be opened, stretched around the second leg of the pair of leggings, and closed around the second leg while worn by the user.

4. The wearable exercise system of claim 1, further comprising a layered stretchable resistance band being attached to the pair of leggings under the stretchable resistance band, the layered stretchable resistance band configured to opened, stretched around the second leg of the pair of leggings, and closed around the second leg while worn by the user.

5. The wearable exercise system of claim 1, comprising a plurality of stretchable resistance bands attached around the first leg of the pair of leggings.

6. The wearable exercise system of claim 1, wherein the first leg is a right-side leg and the second leg is a left side leg.

7. The wearable exercise system of claim 1, wherein the first leg is a left side leg and the second leg is a right-side leg.

8. The wearable exercise system of claim 1, wherein a vertical section of the stretchable resistance band is attached with a seam to the pair of leggings.

9. The wearable exercise system of claim 1, wherein the closure mechanism is a zipper.

10. The wearable exercise system of claim 1, wherein the pair of leggings further comprises at least one legging loop, wherein one band of the stretchable resistance band fits through the legging loop.

11. The wearable exercise system of claim 1, further comprising at least one pocket attached to the pair of leggings, the pocket configured to hold a weight.
